(19) United States
(12) Patent Application Publication
(10) Pub. No.: US 2020/0234813 A1
(43) Pub. Date: Jul. 23, 2020

(54) MULTI-DISCIPLINARY CLINICAL EVALUATION IN VIRTUAL OR AUGMENTED REALITY

(71) Applicant: XR Health IL LTD, Tel Aviv (IL)

(72) Inventor: Eran Orr, Brookline, MA (US)

(21) Appl. No.: 16/839,349

(22) Filed: Apr. 3, 2020

Related U.S. Application Data
(63) Continuation of application No. PCT/US18/54667, filed on Oct. 5, 2018.
(60) Provisional application No. 62/568,721, filed on Oct. 5, 2017.

(51) Int. Cl. G16H 20/30; G16H 50/20; G16H 50/30

(52) U.S. Cl. CPC G16H 20/30; G06T 19/006; G16H 50/30; G16H 50/20

(57) ABSTRACT

Continuous clinical evaluation and care adjustment in virtual reality (VR) or augmented reality (AR) environments is provided. In various embodiments, an evaluation protocol is read from a datastore. The evaluation protocol comprises a plurality of tasks. Each of the plurality of tasks are presented to a user via a virtual or augmented reality display. Positional data are collected from a plurality of sensors. The positional data is received at a remote server and compared to the evaluation protocol to determine a score reflecting the clinical evaluation of the user based on the performance of the plurality of tasks. Whether the score differs from a predetermined threshold is determined at the remote server. A healthcare regimen is adjusted when the score differs from the threshold.

### CROSS-REFERENCE TO RELATED APPLICATIONS

This application claims the benefit of U.S. Provisional Application No. 62/568,721 filed Oct. 5, 2017, which is hereby incorporated by reference in its entirety.

### BACKGROUND

Embodiments of the present disclosure relate to continuous clinical evaluation and care adjustment using virtual or augmented reality, and more specifically, to performing a variety of assessments of cognitive or physical performance through virtual environments and adjusting care of a patient based on the results of the assessment(s).

### BRIEF SUMMARY

According to embodiments of the present disclosure, systems for, methods of, and computer program products for continuous clinical evaluation and care adjustment are provided. In various embodiments, a virtual environment is provided to a user via a virtual or augmented reality system. The virtual or augmented reality system includes a head-mounted display. An evaluation protocol is read from a datastore. The evaluation protocol comprises a plurality of tasks. Each of the plurality of tasks are presented to a user via the virtual or augmented reality display. Positional data are collected of the user. Collecting positional data includes collecting positional data of the head-mounted display. The positional data is received at a remote server. The positional data is compared at the remote server to the evaluation protocol to determine a score. The score reflects a clinical evaluation of the user based on the performance of the plurality of tasks. Whether the score differs from a predetermined threshold is determined at the remote server. A healthcare regimen is adjusted when the score differs from the threshold.

In various embodiments, a system includes a datastore, a virtual or augmented reality display adapted to display a virtual environment to a user, a plurality of sensors coupled to the user, and a computing node comprising a computer readable storage medium having program instructions embodied therewith. The processor of the computing node executes the program instructions to cause the processor to perform a method where a virtual environment is provided to a user via a virtual or augmented reality system. The virtual or augmented reality system includes a head-mounted display an evaluation protocol is read from a datastore. The evaluation protocol comprises a plurality of tasks. Each of the plurality of tasks are presented to a user via the virtual or augmented reality display. Positional data are collected of the user. Collecting positional data includes collecting positional data of the head-mounted display. The positional data is received at a remote server. The positional data is compared at the remote server to the evaluation protocol to determine a score. The score reflects a clinical evaluation of the user based on the performance of the plurality of tasks. Whether the score differs from a predetermined threshold is determined at the remote server. A healthcare regimen is adjusted when the score differs from the threshold.

In various embodiments, a computer program product for continuous clinical evaluation and care adjustment includes a computer readable storage medium having program instructions embodied therewith. The program instructions are executable by a processor to cause the processor to perform a method where a virtual environment is provided to a user via a virtual or augmented reality system. The virtual or augmented reality system includes a head-mounted display an evaluation protocol is read from a datastore. The evaluation protocol comprises a plurality of tasks. Each of the plurality of tasks are presented to a user via the virtual or augmented reality display. Positional data are collected of the user. Collecting positional data includes collecting positional data of the head-mounted display. The positional data is received at a remote server. The positional data is compared at the remote server to the evaluation protocol to determine a score. The score reflects a clinical evaluation of the user based on the performance of the plurality of tasks. Whether the score differs from a predetermined threshold is determined at the remote server. A healthcare regimen is adjusted when the score differs from the threshold.

### BRIEF DESCRIPTION OF THE SEVERAL VIEWS OF THE DRAWINGS

FIGS. 1A-1C illustrates exemplary ADL and IADL questionnaires according to embodiments of the present disclosure.

FIG. 2 illustrates an exemplary virtual reality headset according to embodiments of the present disclosure.

FIG. 3 illustrates an exemplary system for clinical evaluation according to embodiments of the present disclosure.

FIG. 4 illustrates an exemplary method for clinical evaluation according to embodiments of the present disclosure.

FIG. 5 illustrates an exemplary method for continuous clinical evaluation and care adjustment according to embodiments of the present disclosure.

FIG. 6 depicts an exemplary computing node according to embodiments of the present invention.

### DETAILED DESCRIPTION

Clinical evaluation of a patient's cognitive and physical functionality generally relies on a series of subjective factors. A variety of test suites and rubrics are available to subjectively determine a patient's cognitive and physical functionality, covering a variety of functional areas.

For example, various rubrics are available for the evaluation of Activities of Daily Living (ADL) and Instrumental Activities of Daily Living (IADL). In general, ADL evaluation includes various questions that lead to evaluation of identified areas of critical function. Problem areas may be identified, which assists in targeting interventions. For example, a simple ADL evaluation may include questions on whether a subject is able, on their own, to dress, feed themselves, prepare meals, walk, get in and out of a bed/chair, use the toilet, bathe themselves, and/or perform personal hygiene. A simple IADL evaluation may include questions on whether a subject is able to cook, clean, do laundry, shop, use the telephone, access means of transportation, take medicines, or manage money. The loss of independence in the performance of an ADL or IADL may be indicative of a chronic illness such as dementia.

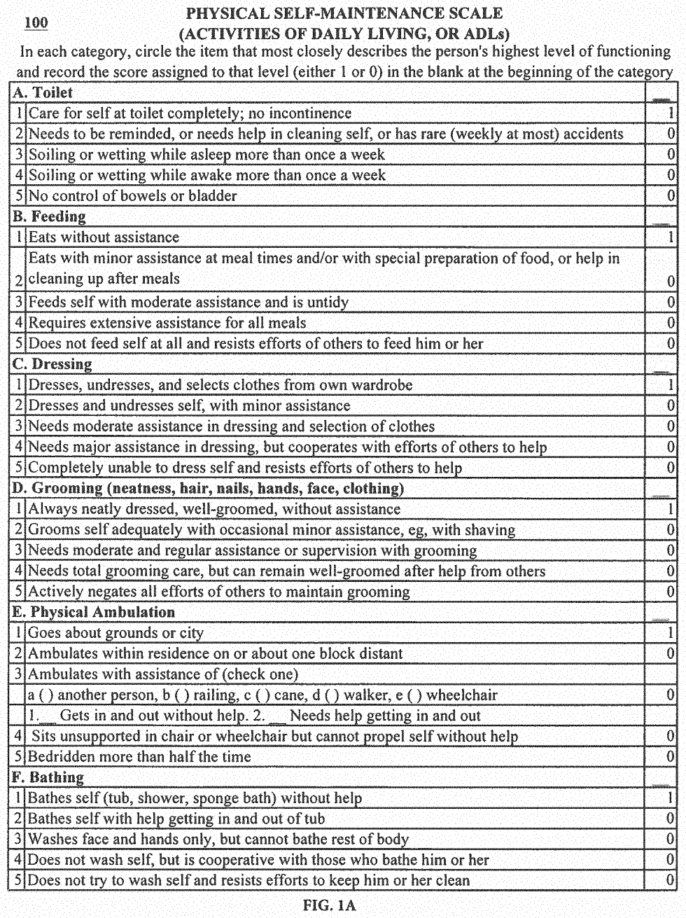

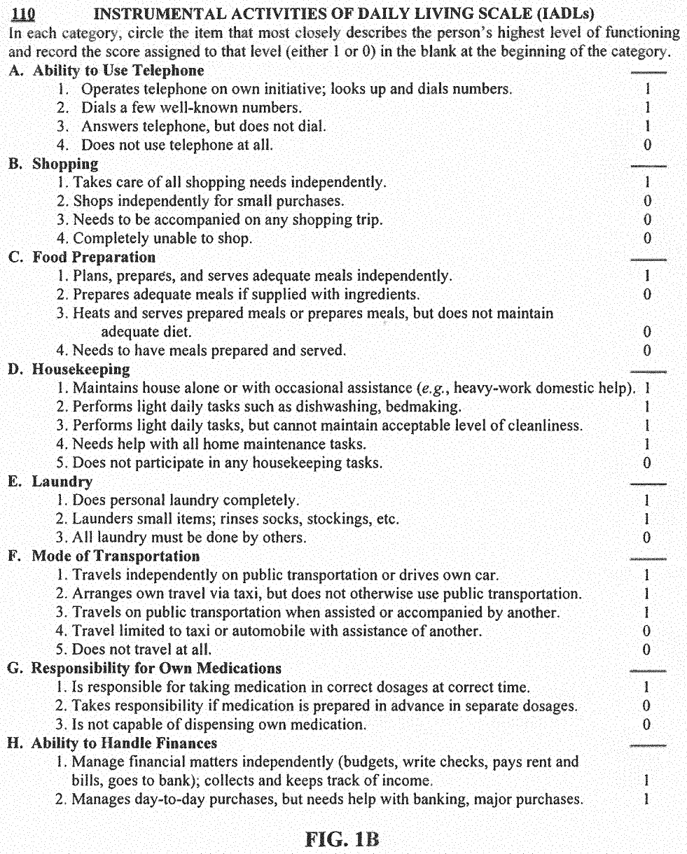

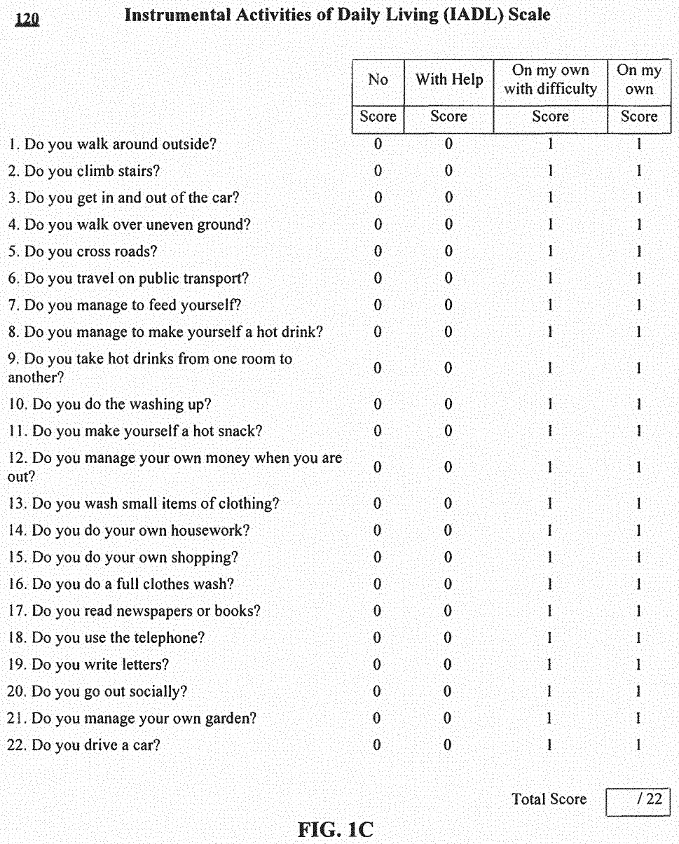

FIG. 1A illustrates an exemplary ADL questionnaire 100 having a list of questions related to whether a patient is capable of performing various routine daily activities. FIGS. 1B and 1C illustrates exemplary IADL questionnaires 110, 120 having a list of questions related to whether a patient is capable of performing various daily activities. If the patient cannot perform the activity or requires assistance to perform the activity, the patient may receive a score of, for example, a zero. If the patient can perform the activity with difficulty, the patient may receive a higher score, e.g., a one. In the patient is capable of performing the activity on their own without difficulty, the patient receive the same score as if they had difficulty performing the activity (e.g., a one), or the patient may receive a higher score (e.g., a two). The patient may be instructed to sum the numerical values received for each question and record the total score. If the total score is higher than a predetermined number, the patient may be classified as capable of performing daily activities without assistance. If the total score is lower than a predetermined number, the patient may be classified as requiring assistance (e.g., from a home health aide or family member) with some or all daily activities. An exemplary form may be found in “Instrumental Activities of Daily Living (ADL) Scale.” Occasional paper (Royal College of General Practitioners) 59 (1993): 25 (which is incorporated by reference herein in its entirety). Another exemplary ADL and/or IADL questionnaire may be found in Lawton M P, Brody E M. “Assessment of older people: self-maintaining and instrumental activities of daily living.” Gerontologist 1969, 9:179-186 (which is incorporated by reference herein in its entirety).

Generally available clinical assessment tools such as ADL and/or IADL questionnaires are not detailed, accurate, accessible, or sufficiently objective. These ADL and/or IADL questionnaires rely on the patient's own subjective opinion (or the opinion of another, e.g., a relative or caretaker) as to the patient's ability to perform certain daily tasks and, thus, do not necessarily provide an accurate assessment of the patient's ability. More accurate alternatives including detailed measurement tools may be prohibitively expensive and/or extremely large, making them inaccessible for most clinics or home use.

Accordingly, there is a need for devices, systems, and methods that facilitate clinical evaluation, such as of ADL, in a portable, cost-effective, and objective manner.

The present disclosure provides for a multi-disciplinary clinical evaluation of a subject using virtual or augmented reality. Using virtual and/or augmented reality to drive clinical assessments allows the creation of fully immersive environments that enables objective patient evaluation of performance of different tasks in different situations. In this way, a subject/patient may engage in simulated tasks, while the system monitors the patient. Immediate feedback may be provided to the patient, various healthcare providers, and/or various healthcare payers (e.g., insurance companies, government agencies, etc.). In various embodiments, the healthcare provider may include a home-health aide, nursing home, hospital, primary care physician, rehabilitation center and/or pharmacy.

In various embodiments, the patient may be provided an evaluation protocol through the VR/AR system to establish a baseline regarding their ability to perform certain daily tasks, for example, tasks taken from the ADL and/or IDL questionnaires described above. In various embodiments, the VR/AR system may read the evaluation protocol from a remote server. In various embodiments, the remote server may include an electronic health record (EHR) database.

In various embodiments, the VR/AR system may record data (e.g., positional, biometric, etc.) as the user performs the presented tasks and compare the recorded data to a predetermined baseline. In various embodiments, the predetermined baseline may include standard clinical guidelines. In various embodiments, the predetermined baseline may include a statistic (e.g., average, standard deviation, variance, etc.) from a sample of patients. In various embodiments, the predetermined baseline may be determined from an initial assessment of the patient at the beginning of, or at any point during, care. In various embodiments, the recorded data may be sent to the remote server for processing and/or storage in a database.

In various embodiments, the recorded data may be compared to the predetermined baseline for similarity. In various embodiments, the predetermined baseline may be stored at the remote server. In various embodiments, the comparison for similarity may include any suitable comparison, such as, for example, comparing averages within a suitable margin of error.

In various embodiments, the recorded data may be processed to determine a quantitative (e.g., integers 0 through 10, etc.) or qualitative (e.g., ‘A’, ‘B’, ‘C’, ‘D’, etc.) score. In various embodiments, the score may be compared to a predetermined score from the evaluation protocol. If the score is similar to the predetermined score from the evaluation protocol, no action may be taken with respect to the patient's healthcare regimen. If the score differs from the predetermined score from the evaluation protocol, an adjustment may be made to a healthcare regimen provided to the patient. In various embodiments, health care providers can be allocated in an efficient manner when and where they are most needed to provide healthcare services to patients.

If the score is lower than the predetermined score from the evaluation protocol, an adjustment may be made to the patient's healthcare regimen. For example, a notification may be provided to one or more healthcare stakeholders (e.g., healthcare provider and/or insurance company) that the patient requires additional medical assistance to complete routine daily activities. In this example, a home-health aide may increase the number of visits to the patient per week, increase the time spent with the patient, and/or provide assistance with additional daily activities where no assistance was previously provided.

If the score is higher than the predetermined score from the evaluation protocol, an adjustment may be made to the patient's healthcare regimen. For example, a notification may be provided to one or more healthcare stakeholders (e.g., healthcare provider and/or insurance company) that the patient does not require as much medical assistance to complete routine daily activities as they have been previously receiving. In this example, a home- health aide may decrease the number of visits to the patient per week, decrease the time spent with the patient, and/or provide assistance with fewer daily activities where assistance was previously provided.

In various embodiments, the adjustment may be recorded at the remote server, for example, in the EHR database. The systems and methods of the disclosure thus allow for easy and accessible auditing of a patient's health care services. Such a system may be useful to the various stakeholders in a patient's healthcare, such as, for example, healthcare providers, insurance companies, and/or governmental agencies to justify reimbursement of healthcare services by providing an objective assessment of a patient's abilities and objective reasoning for adjustments in the patient's healthcare.

In various embodiments, the patient may repeat the assessment for a predetermined number of times and/or on a predetermined schedule (e.g., weekly, biweekly, monthly, etc.). In various embodiments, future assessments may be compared to previous assessments and/or baseline values to thereby determine whether a level of care should be continued or adjusted.

In various embodiments, data and/or scores from updated assessments may be used to determine an updated baseline value. For example, data and/or scores from many different patients may be used to update the baseline statistics for use with future patients and/or future assessments.

Such an integrated VR/AR platform enables reduction of costs, improvement in objectivity, and allows for highly accurate measurements with fully detailed outputs to the various stakeholders in a patient's healthcare. The solutions provided herein provide portable and accessible tools that can be used both in clinical settings and at a patient's home. It will be appreciated that although the present disclosure describes several ADL examples, the present disclosure is applicable to a variety of clinical assessment use cases.

It will be appreciated that a variety of virtual and augmented reality devices are known in the art. For example, various head-mounted displays providing either immersive video or video overlays are provided by various vendors. Some such devices integrate a smart phone within a headset, the smart phone providing computing and wireless communication resources for each virtual or augmented reality application. Some such devices connect via wired or wireless connection to an external computing node such as a personal computer. Yet other devices may include an integrated computing node, providing some or all of the computing and connectivity required for a given application.

Virtual or augmented reality displays may be coupled with a variety of motion sensors in order to track a user's motion within a virtual environment. Such motion tracking may be used to navigate within a virtual environment, to manipulate a user's avatar in the virtual environment, or to interact with other objects in the virtual environment. In some devices that integrate a smartphone, head tracking may be provided by sensors integrated in the smartphone, such as an orientation sensor, gyroscope, accelerometer, or geomagnetic field sensor. Sensors may be integrated in a headset, or may be held by a user, or attached to various body parts (e.g., a limb and/or chest) to provide detailed information on user positioning.

In various embodiments, the VR/AR system may determine the position of the body part and record the position over time. In various embodiments, as described in more detail above, one or more sensors may be attached to or otherwise associated with a body part to track a three-dimensional position and motion of the body part with six degrees of freedom. In various embodiments, the system may determine a plurality of positions of one or more body parts. The plurality of positions may correspond to points along a three-dimensional path taken by the body part.

In various embodiments, the system may track the position and motion of the head. In various embodiments, the system may utilize sensors in a head-mounted display to determine the position and motion of the head with six degrees of freedom as described below. Head tracking may be implemented in various embodiments where position/motion data may be compared to an evaluation protocol to determine, through a quantitative metric (e.g., number) or qualitative metric (e.g., color scale), how accurately the patient is performing a presented task. For example, head tracking may be implemented when using an evaluation protocol that includes routine daily activities (e.g., getting out of bed, getting dressed, etc.).

In various embodiments, for more nuanced exercises/activities, one or more additional sensors may provide position/motion data of various body parts.

In various embodiments, additional sensors are included to measure characteristics of a subject in addition to motion. For example, cameras and microphones may be included to track speech, eye movement, blinking rate, breathing rate, and facial features. Biometric sensors may be included to measure features such as heart rate (pulse), inhalation and/or exhalation volume, perspiration, eye blinking rate, electrical activity of muscles, electrical activity of the brain or other parts of the central and/or peripheral nervous system, blood pressure, glucose, temperature, galvanic skin response, or any other suitable biometric measurement as is known in the art.

In various embodiments, an electrocardiogram (EKG) may be used to measure heart rate. In various embodiments, an optical sensor may be used to measure heart rate, for example, in a commercially-available wearable heart rate monitor device. In various embodiments, a wearable device may be used to measure blood pressure separately from or in addition to heart rate. In various embodiments, a spirometer may be used to measure inhalation and/or exhalation volume. In various embodiments, a humidity sensor may be used to measure perspiration. In various embodiments, a camera system may be used to measure the blinking rate of one or both eyes. In various embodiments, a camera system may be used to measure pupil dilation. In various embodiments, an electromyogram (EMG) may be used to measure electrical activity of one or more muscles. The EMG may use one or more electrodes to measure electrical signals of the one or more muscles. In various embodiments, an electroencephalogram (EEG) may be used to measure electrical activity of the brain. The EEG may use one or more electrodes to measure electrical signals of the brain. Any of the exemplary devices listed above may be connected (via wired or wireless connection) to the VR/AR systems described herein to thereby provide biometric data/measurements for analysis. In various embodiments, breathing rate may be measured using a microphone.

In various embodiments, a user is furnished with a VR or AR system. As noted above, a VR or AR system will generally have integrated motion sensors. In addition, additional motions sensors may be provided, for example to be handheld. This allows tracking of multiple patient attributes while they interact with a scene. In this way, systematic and reproducible scenarios may be used to assess the subject's function.

In particular, an assessment protocol may be presented to a user while they are immersed in a virtual or augmented reality environment. For example, a fine motor task may be presented, and then reaction time and precision measured. In another example, a puzzle is displayed, and completion time or hesitation is measured.

In various embodiments, patient motion may be tracked. For example, Gait, Stability, Tremor, Amplitude of Motion, Speed of Motion, and Range of Motion may be measured. Movement may be analyzed to determine additional second order attributes such as smoothness or rigidity.

In various embodiments, cognitive ability may be tracked, for example by presentation of a cognitive challenge. For example, Reaction time, Success rate in cognitive challenges, Task fulfillment under verbal, written, or illustrated guidance, Understanding of instructions, Memory performance, Social interaction, and Problem solving may be measured.

In various embodiments, speech attributes are tracked. For example, Fluency of Speech, Ability to imitate, and Pronunciation are assessed. It will be appreciated that any of the tests described herein, may be performed in a variety of languages according to the needs of a given patient.

In various embodiments, overall stability and stance may be tracked.

In various embodiments, facial expressions may be tracked. For example, particular expressions may be recognized.

In various embodiments, additional biometrics may be measured.

In various embodiments, fatigue is assessed. For example, reaction time, attention, and hand-eye coordination may be assessed as set forth above. In aggregate, these factors may be used to measure overall fatigue.

The tracking of these metrics allows the generation of quantified, detailed reports that are aligned with common practice evaluation procedures. It will be appreciated that a variety of evaluation protocol are known in the art. By way of illustration and not limitation, the present disclosure may be used to conduct walking tests, Timed Up and Go (TUG) tests, the Montreal Cognitive Assessment (MOCA), functional reach tests, the Mini-Mental State Examination (MMSE), or any of a variety of other evaluations. It will be appreciated that these various measures may be compared to clinical guidance to assist in diagnosis.

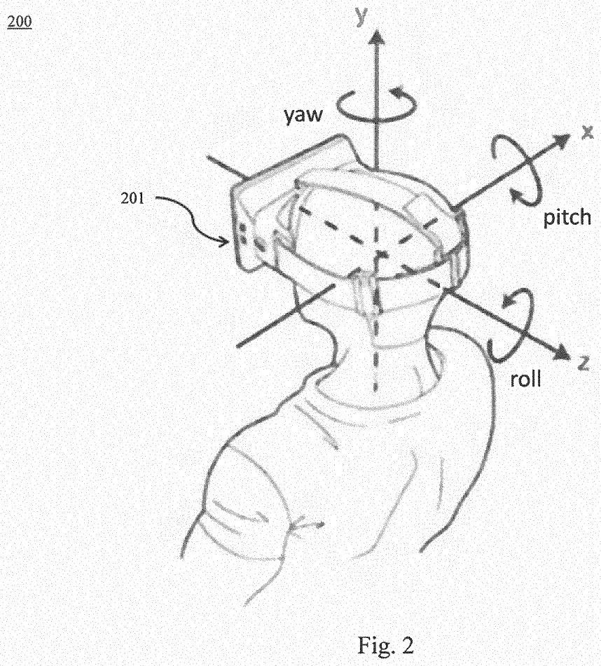

With reference now to FIG. 2, an exemplary virtual reality headset is illustrated according to embodiments of the present disclosure. In various embodiments, system 200 is used to collected data from motion sensors including hand sensors (not pictured), sensors included in headset 201, and additional sensors such as torso sensors or a stereo camera. In some embodiments, data from these sensors is collected at a rate of up to about 150 Hz. As pictured, data may be collected in six degrees of freedom: X-left/right; Y-up/down/height; Z-foreword/backward; P-pitch; R-roll; Y-yaw. As set out herein, this data may be used to track a user's overall motion to facilitate interaction with a virtual environment and to evaluate their performance.

It will be appreciated that different modes of interaction may be appropriate for administering different clinical evaluations. By way of illustration, several are provided below. However, many other potential modes of interaction will be recognized in view of the present disclosure.

For cognitive tests that require drawing, tracing, or following an object of sequence of objects, the user in a virtual environment may gesture with their hand to draw or trace as appropriate. For example, in tests requiring copying a drawing, a user may be shown a form suspended in space, and then directed to use a virtual pen to reproduce the form. The degree of accuracy may be measured and reported.

For cognitive tests that require naming people, animals, or things, the subject of identification may be displayed to the user in the virtual environment. The user may select their response from an in-environment menu, or speak their answer to be detected via speech recognition. The accuracy, and any hesitation or stuttering may be measured.

For tests that require repetition of words or patterns (e.g., as in the Simon game), a pattern may be displayed to a user for them to reproduce by speaking, or by gesturing in the virtual environment. Accuracy, and response time may be measured.

For tests that evaluate spatial awareness and dexterity, a moving 3D character or scene may be displayed in the virtual space around the subject, guiding the subject's motions. The subject's accuracy, mobility, response time, and stamina may be measured.

For tests that evaluate stance and balance, sway assessment may be performed. In various embodiments, the sway may be calculated based on sensor feedback from handheld (or otherwise hand-affixed) sensors and from head mounted sensors. Changing scenery may be presented in order to manipulate the visual & vestibular systems in order to get a comprehensive result. In this way, balance may be measured.

For tests that evaluate other biometric data, additional sensors are used. Biometric data may reflect the patient's physiological or psychological state, indicating functioning of the body systems or cognition under the stimulus of a given virtual environment. In various embodiments, sensors connected to the user provide: Heart rate variability (HRV); Electrothermal activity (EDA); Galvanic skin response (GSR); Electroencephalography (EEG); Electromyography (EMG); Eye tracking; Electrooculography (EOG); Patient's range of motion (ROM); Patient's velocity performance; Patient's acceleration performance; or Patient's smoothness performance.

For tests that evaluate memory, processing speed, or problem-solving skills, a user may be presented with a virtual puzzle For example, a maze.

In various embodiments, a library of predetermined evaluation tasks is maintained. To provide a comprehensive evaluation, the tasks may be combined. The comprehensive evaluation may correspond to a known evaluation procedure, or may form a superset or subset of a known procedure. In this way, multiple standard tests may be applied without duplication of tasks.

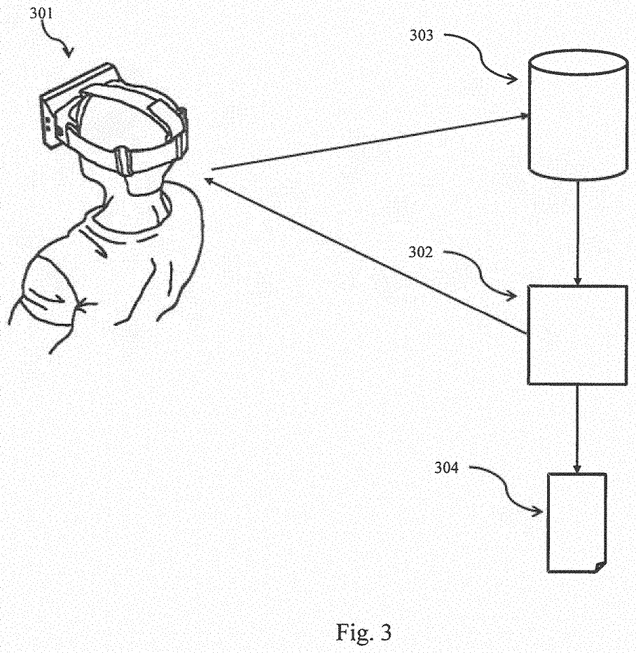

Referring to FIG. 3, an exemplary system according to the present disclosure is illustrated. A patient is connected to VR headset 301. It will be appreciated that a variety of alternative VR or AR devices are suitable for use according to the present disclosure. Likewise, as noted above, a variety of sensors may be connected to the patient to provide a broader variety of data than are available from a headset alone. Headset 301 receives an appropriate VR environment from system 302. In some embodiments, system 302 is a remote server, while in some embodiments system 302 is a local computer. The VR environment is displayed to the user, and data is collected while the user performs the appropriate tasks.

User data are stored in data store 303. In some embodiments, datastore 303 is a remote database. Data are provided to system 302 for report generation. In various embodiments, report templates are provided by system 302 that correspond to various known evaluation rubrics. The data drawn from datastore 303 are used to populate a given template, and a report 304 is generated.

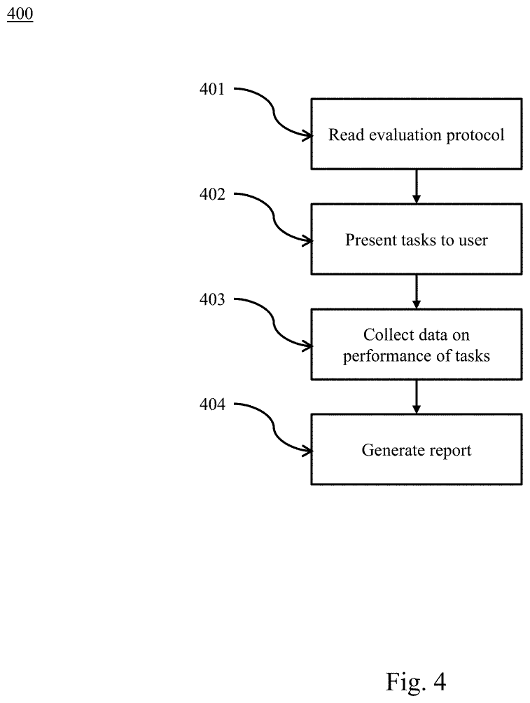

Referring now to FIG. 4, a method of clinical evaluation according to embodiments of the present disclosure is illustrated. At 401, an evaluation protocol is read from a datastore. The evaluation protocol comprises a plurality of tasks. At 402, the plurality of tasks are presented to a user via a virtual or augmented reality display. At 403, data are collected from a plurality of sensors regarding the user's performance of the plurality of tasks. At 404, a report is generated reflecting the clinical evaluation of the user based on the performance of the plurality of tasks.

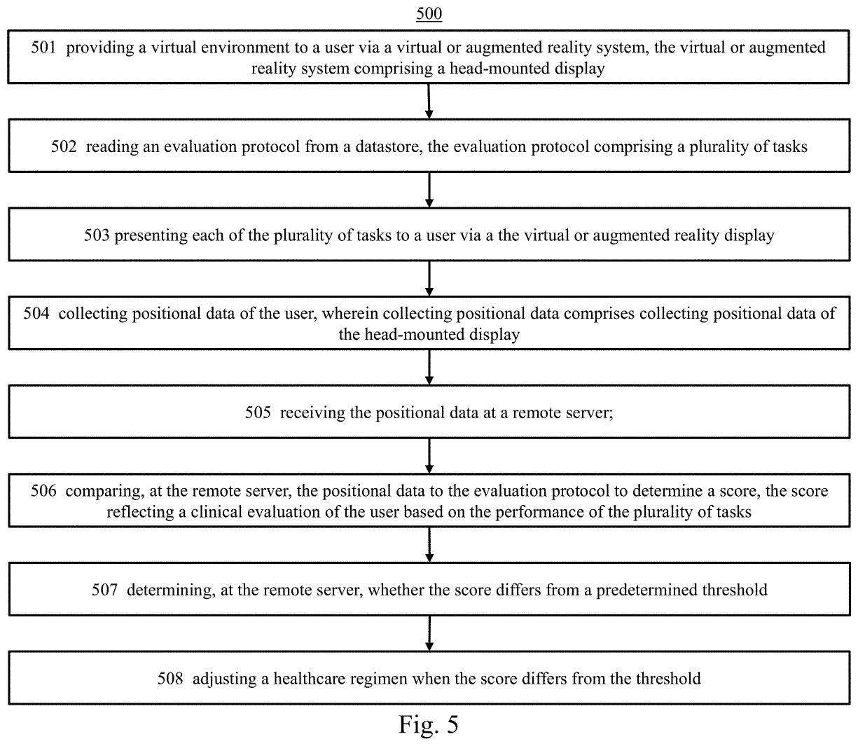

Referring now to FIG. 5, a method 500 of continuous clinical evaluation and care adjustment according to embodiments of the present disclosure is illustrated. At 501, a virtual environment is provided to a user via a virtual or augmented reality system. At 502, an evaluation protocol is read from a datastore. The evaluation protocol comprises a plurality of tasks. At 503, each of the plurality of tasks are presented to a user via the virtual or augmented reality display. At 504, positional data are collected of the user, wherein collecting positional data comprises collecting positional data of the head-mounted display. At 505, the positional data is received at a remote server. At 506, the positional data is compared at the remote server to the evaluation protocol to determine a score. The score reflects a clinical evaluation of the user based on the performance of the plurality of tasks. At 507, whether the score differs from a predetermined threshold is determined at the remote server. At 508, a healthcare regimen is adjusted when the score differs from the threshold.

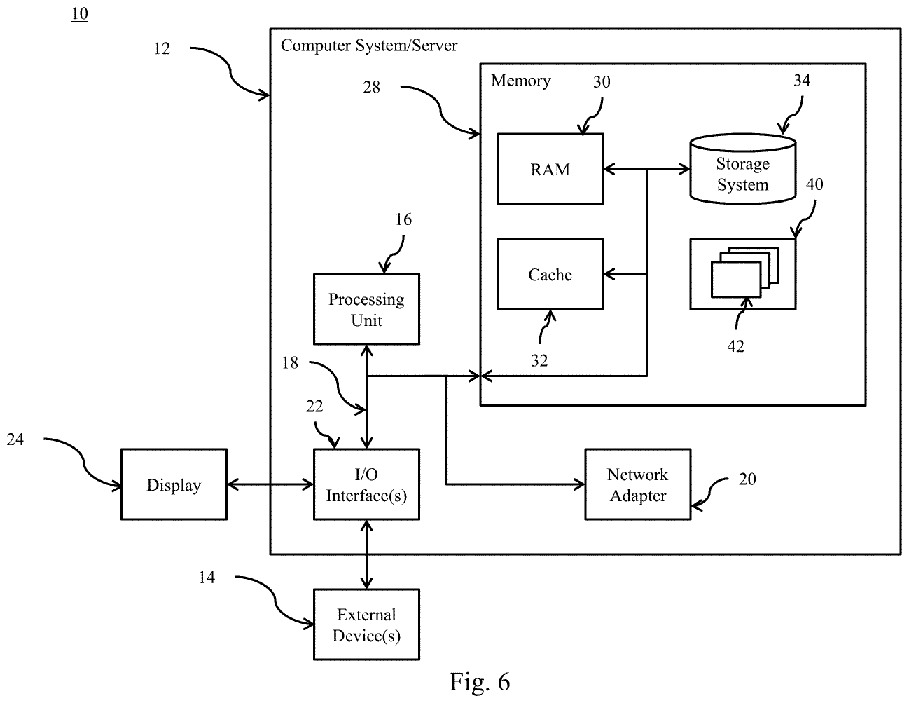

Referring now to FIG. 6, a schematic of an example of a computing node is shown. Computing node 10 is only one example of a suitable computing node and is not intended to suggest any limitation as to the scope of use or functionality of embodiments of the invention described herein. Regardless, computing node 10 is capable of being implemented and/or performing any of the functionality set forth hereinabove.

In computing node 10 there is a computer system/server 12, which is operational with numerous other general purpose or special purpose computing system environments or configurations. Examples of well-known computing systems, environments, and/or configurations that may be suitable for use with computer system/server 12 include, but are not limited to, personal computer systems, server computer systems, thin clients, thick clients, handheld or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like.

Computer system/server 12 may be described in the general context of computer system-executable instructions, such as program modules, being executed by a computer system. Generally, program modules may include routines, programs, objects, components, logic, data structures, and so on that perform particular tasks or implement particular abstract data types. Computer system/server 12 may be practiced in distributed cloud computing environments where tasks are performed by remote processing devices that are linked through a communications network. In a distributed cloud computing environment, program modules may be located in both local and remote computer system storage media including memory storage devices.

As shown in FIG. 6, computer system/server 12 in computing node 10 is shown in the form of a general-purpose computing device. The components of computer system/server 12 may include, but are not limited to, one or more processors or processing units 16, a system memory 28, and a bus 18 that couples various system components including system memory 28 to processor 16.

Bus 18 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus.

Computer system/server 12 typically includes a variety of computer system readable media. Such media may be any available media that is accessible by computer system/server 12, and it includes both volatile and non-volatile media, removable and non-removable media.

System memory 28 can include computer system readable media in the form of volatile memory, such as random access memory (RAM) 30 and/or cache memory 32. Computer system/server 12 may further include other removable/non-removable, volatile/non-volatile computer system storage media. By way of example only, storage system 34 can be provided for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 18 by one or more data media interfaces. As will be further depicted and described below, memory 28 may include at least one program product having a set (e.g., at least one) of program modules that are configured to carry out the functions of embodiments of the invention.

Program/utility 40, having a set (at least one) of program modules 42, may be stored in memory 28 by way of example, and not limitation, as well as an operating system, one or more application programs, other program modules, and program data. Each of the operating system, one or more application programs, other program modules, and program data or some combination thereof, may include an implementation of a networking environment. Program modules 42 generally carry out the functions and/or methodologies of embodiments of the invention as described herein.

Computer system/server 12 may also communicate with one or more external devices 14 such as a keyboard, a pointing device, a display 24, etc.; one or more devices that enable a user to interact with computer system/server 12; and/or any devices (e.g., network card, modem, etc.) that enable computer system/server 12 to communicate with one or more other computing devices. Such communication can occur via Input/Output (I/O) interfaces 22. Still yet, computer system/server 12 can communicate with one or more networks such as a local area network (LAN), a general wide area network (WAN), and/or a public network (e.g., the Internet) via network adapter 20. As depicted, network adapter 20 communicates with the other components of computer system/server 12 via bus 18. It should be understood that although not shown, other hardware and/or software components could be used in conjunction with computer system/server 12. Examples, include, but are not limited to: microcode, device drivers, redundant processing units, external disk drive arrays, RAID systems, tape drives, and data archival storage systems, etc.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD- ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

A Picture Archiving and Communication System (PACS) is a medical imaging system that provides storage and access to images from multiple modalities. In many healthcare environments, electronic images and reports are transmitted digitally via PACS, thus eliminating the need to manually file, retrieve, or transport film jackets. A standard format for PACS image storage and transfer is DICOM (Digital Imaging and Communications in Medicine). Non-image data, such as scanned documents, may be incorporated using various standard formats such as PDF (Portable Document Format) encapsulated in DICOM.

An electronic health record (EHR), or electronic medical record (EMR), may refer to the systematized collection of patient and population electronically-stored health information in a digital format. These records can be shared across different health care settings and may extend beyond the information available in a PACS discussed above. Records may be shared through network-connected, enterprise-wide information systems or other information networks and exchanges. EHRs may include a range of data, including demographics, medical history, medication and allergies, immunization status, laboratory test results, radiology images, vital signs, personal statistics like age and weight, and billing information.

EHR systems may be designed to store data and capture the state of a patient across time. In this way, the need to track down a patient's previous paper medical records is eliminated. In addition, an EHR system may assist in ensuring that data is accurate and legible. It may reduce risk of data replication as the data is centralized. Due to the digital information being searchable, EMRs may be more effective when extracting medical data for the examination of possible trends and long term changes in a patient. Population-based studies of medical records may also be facilitated by the widespread adoption of EHRs and EMRs.

Health Level-7 or HL7 refers to a set of international standards for transfer of clinical and administrative data between software applications used by various healthcare providers. These standards focus on the application layer, which is layer 7 in the OSI model. Hospitals and other healthcare provider organizations may have many different computer systems used for everything from billing records to patient tracking. Ideally, all of these systems may communicate with each other when they receive new information or when they wish to retrieve information, but adoption of such approaches is not widespread. These data standards are meant to allow healthcare organizations to easily share clinical information. This ability to exchange information may help to minimize variability in medical care and the tendency for medical care to be geographically isolated.

In various systems, connections between a PACS, Electronic Medical Record (EMR), Hospital Information System (HIS), Radiology Information System (RIS), or report repository are provided. In this way, records and reports form the EMR may be ingested for analysis. For example, in addition to ingesting and storing HL7 orders and results messages, ADT messages may be used, or an EMR, RIS, or report repository may be queried directly via product specific mechanisms. Such mechanisms include Fast Health Interoperability Resources (FHIR) for relevant clinical information. Clinical data may also be obtained via receipt of various HL7 CDA documents such as a Continuity of Care Document (CCD). Various additional proprietary or site-customized query methods may also be employed in addition to the standard methods.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

1. A method comprising:
providing a virtual environment to a user via a virtual or augmented reality system, the virtual or augmented reality system comprising a head-mounted display;
reading an evaluation protocol from a datastore, the evaluation protocol comprising a plurality of tasks;
presenting each of the plurality of tasks to a user via the virtual or augmented reality display;
collecting positional data of the user, wherein collecting positional data comprises collecting positional data of the head-mounted display;
receiving the positional data at a remote server;
comparing, at the remote server, the positional data to the evaluation protocol to determine a score, the score reflecting a clinical evaluation of the user based on the performance of the plurality of tasks;
determining, at the remote server, whether the score differs from a predetermined threshold; and
adjusting a healthcare regimen when the score differs from the threshold.

2. The method of claim 1, wherein the datastore comprises an electronic health record.

3. The method of claim 2, further comprising recording an adjustment from adjusting the healthcare regimen in the electronic health record.

4. The method of claim 3, further comprising sending the adjustment to a healthcare payer.

5. The method of claim 1, wherein adjusting the healthcare regimen comprises notifying a healthcare provider of an adjustment.

6. The method of claim 5, wherein the adjustment comprises an increase in number of visits from a healthcare provider.

7. The method of claim 5, wherein the adjustment comprises a decrease in number of visits from a healthcare provider.

8. The method of claim 1, further comprising collecting biometric data from the user.

9. The method of claim 8, wherein the biometric data comprises eye movement collected with a camera.

10. (canceled)

11. The method of claim 8, wherein the biometric data comprises breathing rate collected with a microphone.

12. (canceled)

13. The method of claim 1, wherein the score is an updated score, the method further comprising:
determining a baseline score corresponding to a baseline health care regimen;
comparing the updated score to the baseline score; and
adjusting the healthcare regimen when the updated score differs from the baseline score.

14. A system comprising:
a datastore;
a virtual or augmented reality display adapted to display a virtual environment to a user;
a plurality of sensors coupled to the user;
a computing node comprising a computer readable storage medium having program instructions embodied therewith, the program instructions executable by a processor of the computing node to cause the processor to perform a method comprising:
providing a virtual environment to a user via a virtual or augmented reality system, the virtual or augmented reality system comprising a head-mounted display;
reading an evaluation protocol from the datastore, the evaluation protocol comprising a plurality of tasks;
presenting each of the plurality of tasks to the user via the virtual or augmented reality display;
collecting positional data of the user, wherein collecting positional data comprises collecting positional data of the head-mounted display;
receiving the positional data at a remote server;
comparing, at the remote server, the positional data to the evaluation protocol to determine a score, the score reflecting a clinical evaluation of the user based on the performance of the plurality of tasks;
determining, at the remote server, whether the score differs from a predetermined threshold; and
adjusting a healthcare regimen when the score differs from the threshold.

15-26. (canceled)

27. A computer program product for continuous clinical evaluation and care adjustment, the computer program product comprising a computer readable storage medium having program instructions embodied therewith, the program instructions executable by a processor to cause the processor to perform a method comprising:
reading an evaluation protocol from a datastore, the evaluation protocol comprising a plurality of tasks;
presenting each of the plurality of tasks to a user via a virtual or augmented reality display, the display comprising a head-mounted display;
collecting positional data of the user, wherein collecting positional data comprises collecting positional data of the head-mounted display;
receiving the positional data at a remote server;
comparing, at the remote server, the positional data to the evaluation protocol to determine a score, the score reflecting a clinical evaluation of the user based on the performance of the plurality of tasks;
determining, at the remote server, whether the score differs from a predetermined threshold; and
adjusting a healthcare regimen when the score differs from the threshold.

28. The computer program product of claim 27, wherein the datastore comprises an electronic health record.

29. The computer program product of claim 28, further comprising recording an adjustment from adjusting the healthcare regimen in the electronic health record.

30. (canceled)

31. The computer program product of claim 27, wherein adjusting the healthcare regimen comprises notifying a healthcare provider of an adjustment.

32. (canceled)

33. (canceled)

34. The computer program product of claim 27, further comprising collecting biometric data from the user.

35. The computer program product of claim 34, wherein the biometric data comprises eye movement collected with a camera.

36. (canceled)

37. The computer program product of claim 34, wherein the biometric data comprises breathing rate collected with a microphone.

38. (canceled)

39. The computer program product of claim 27, wherein the score is an updated score, the method further comprising:
determining a baseline score corresponding to a baseline health care regimen;
comparing the updated score to the baseline score; and
adjusting the healthcare regimen when the updated score differs from the baseline score.
